(19) United States
(12) Patent Application Publication
(10) Pub. No.: US 2020/0368448 A1
(43) Pub. Date: Nov. 26, 2020

(54) SYRINGE FILLING ADJUSTMENT DEVICES AND METHODS

(71) Applicant: NeuroDerm, Ltd., Rehovot (IL)

(72) Inventors: Eran Shor, Arugot (IL); Tsabar Mor, Adi (IL); Yael Bentov, Rishon LeZion (IL); Eden Pogiro, Givatayim (IL)

(21) Appl. No.: 16/838,384

(22) Filed: Apr. 2, 2020

Related U.S. Application Data
(60) Provisional application No. 62/851,903, filed on May 23, 2019.

(51) Int. Cl. A61M 5/315; A61M 5/31; A61M 5/168

(52) U.S. Cl. CPC A61M 5/31561; A61M 5/3146; A61M 2005/31516; A61M 5/31528; A61M 5/16877

(57) ABSTRACT

A syringe liquid adjusting device provides a first elongated chamber for reciprocally moving a plunger shaft of a syringe in the syringe barrel to thereby reduce a liquid volume in the syringe barrel from an initial liquid volume to a total liquid volume including a priming liquid volume for priming an infusion line and, in addition, an infusion liquid volume for patient infusion. The syringe liquid adjusting device additionally provides a second elongated chamber for further reciprocally moving the plunger shaft of the syringe in the syringe barrel to further reduce the liquid volume in the syringe barrel from the total liquid volume to the infusion liquid volume while priming the infusion line connected to the syringe with the priming liquid volume. The two elongated chambers may be provided by a one-member, dual-function, device, or by two, separate, members. The one-member device may provide a lengthwise variable elongated chamber.

### CROSS-REFERENCE TO RELATED APPLICATIONS

This application claims priority to co-pending U.S. Provisional Patent Application No. 62/851,903, titled “Syringe Filling Adjustment Devices and Methods,” filed May 23, 2019, the disclosure of which is incorporated herein by reference in its entirety.

### FIELD OF THE INVENTION

The present invention generally relates to devices and methods for adjusting the amount of liquid (e.g., pharmaceutical drug, medicament) in liquid dispensing devices (for example in an infusion system in preparation for infusion delivery to patients), and , by way of example, to devices and methods for adjusting the amount of liquid in an infusion syringe (or another container containing liquid) to an amount of liquid required for, for example, infusion, and optionally also for priming and filling an infusion set via which infusion liquid is transferred from the infusion system to a user being, or to be, infused with the infusion liquid.

### BACKGROUND

Parkinson's disease (PD) patients typically experience a range of motor symptoms during the course of the illness, for example slowness/bradykinesia, tremor, muscle stiffness, dyskinesia, etc.

Some infusion systems include, or use, a syringe, for delivering a drug to PD patients. A drug to be delivered to patients may be delivered via an infusion set, or infusion tube by expelling drug out from the syringe barrel, and into the infusion tube. Typically, using such infusion systems requires manually pre-filling the syringe with an exact prescribed dose of liquid content from a drug vial, and priming the infusion set/tube with a priming liquid. Filling the syringe to the exact prescribed dose (e.g., by using scale marks) and priming the infusion set/tube require very gentle motor skills (e.g., ability to make small, fully controlled, movements, apply a steady force by the fingers, etc.), for which reason handling such a syringe-filling process is oftentimes a very frustrating challenge to PD patients. Filling a syringe filling with a prescribed amount of drug could also be a frustrating challenge to elderly people and to people with relatively poor eyesight or visual acuity who use such infusion systems.

It would be beneficial to have a liquid (e.g., drug) adjusting device for a syringe, and, in general, it would be beneficial to have a liquid adjusting device for a syringe that enables PD patients, and others, to adjust liquid in a syringe in an easier and more simple way.

### SUMMARY OF THE INVENTION

A syringe liquid adjusting device is provided, which provides a first elongated chamber having a length L1 to facilitate reciprocal movement of a plunger shaft of a syringe in the syringe barrel to thereby reduce a liquid volume in the syringe barrel from an initial liquid volume to a total liquid volume including a priming liquid volume for priming an infusion tube and, in addition, an infusion liquid volume for patient infusion. The syringe liquid adjusting device additionally provides a second elongated chamber having a length L2 (L2<L1) to facilitate further reciprocal movement of the plunger shaft in the syringe barrel to reduce the liquid volume in the syringe barrel from the total liquid volume to the infusion liquid volume while priming the infusion tube connected to the syringe with the priming liquid volume.

In some embodiments, the two elongated chambers are provided by a one-member, dual-function, device. The one-member device may provide an elongated chamber whose length is adjustable between length L2 and length L1, or an elongated chamber that provides two, axially spaced apart, positions for the barrel flange—one axial position that corresponds to length L1, and the other axial position that corresponds to length L2.

In other embodiments, the two elongated chambers are provided by two, separate, members, where each member provides an elongated chamber of different length. Also provided are methods for adjusting a liquid volume in a syringe barrel by using the one-member and two-member syringe liquid adjusting devices.

### BRIEF DESCRIPTION OF THE DRAWINGS

Various exemplary embodiments are illustrated in the accompanying figures with the intent that these examples not be restrictive. It will be appreciated that for simplicity and clarity of the illustration, elements shown in the figures referenced below are not necessarily drawn to scale. Also, where considered appropriate, reference numerals may be repeated among the figures to indicate like, corresponding or analogous elements. Of the accompanying figures:

FIG. 1A shows a two-member syringe liquid adjusting device for adjusting a liquid volume in a syringe barrel according to an example embodiment;

FIG. 1B shows a two-member syringe liquid adjusting device for adjusting a liquid volume in a syringe barrel according to another example embodiment;

FIG. 2 depicts a two-member syringe liquid adjusting device for adjusting a liquid volume in a syringe barrel according to an example embodiment;

FIGS. 3A-3F depict an example method of using the two-member syringe liquid adjusting device of, for example, FIG. 2;

FIG. 4 schematically illustrates a single-member, dual function, syringe liquid adjusting device according to an example embodiment;

FIGS. 5A-5C show example locking mechanism for a single-member, dual function, syringe liquid adjusting device according to example embodiments;

FIGS. 6A-6D schematically illustrates a single-member, dual function, syringe liquid adjusting device according to an example embodiment;

FIGS. 7A-7C show example locking mechanisms for a single-member, dual function, syringe liquid adjusting device according to example embodiments;

FIG. 8 shows a method for adjusting a liquid volume in a syringe barrel according to an example embodiment; and

FIG. 9 shows a method for adjusting a liquid volume in a syringe barrel according to another example embodiment.

### DETAILED DESCRIPTION OF THE INVENTION

The description that follows provides various details of exemplary embodiments. However, this description is not intended to limit the scope of the claims but instead to explain various principles of the invention and exemplary manner of practicing it.

Syringe liquid adjustment methods are provided for adjusting the amount of liquid (e.g., drug, medicament) in a syringe barrel. In some embodiments of the present invention, a one-step adjustment method may include adjustment of the liquid volume in the syringe barrel to include an exact prescribed amount of liquid for infusion. In other embodiments of the present invention, the method of adjusting the amount of liquid in a syringe may be a two-step adjustment process that includes a first filling step that includes adjustment of the liquid volume in the syringe to include an exact prescribed amount of liquid for infusion and also an exact amount of liquid for filling out (i.e., priming) an infusion tube connected to the syringe, and a second filling step that includes priming the infusion tube after which the liquid volume remaining in the syringe barrel is the prescribed amount of liquid intended for infusion. (The prescribed amount of infusion liquid that is to be delivered/administered to a patient is referred to herein as “infusion liquid volume”; the exact amount of liquid for completely filling up an infusion tube that is used to deliver the infusion liquid volume is referred to herein as “priming liquid volume”, and a liquid volume including the infusion liquid volume and the priming liquid volume is referred to herein as “total liquid volume”.)

A device for adjusting a liquid volume in a syringe is also provided, which may be used according to any of the syringe liquid adjustment methods described herein or using similar methods. Briefly, in some embodiments, the syringe liquid adjusting device may include one syringe's plunger shaft receiving member (that may be, for example, a cylinder including a blind hole that is designed to receive the plunger shaft), that may be operated using any desired syringe liquid adjusting method (e.g., the one-step adjustment method, or the two-step adjustment method). In some embodiments, the one plunger shaft receiving member may be fixed in length but provide two, distinct, longitudinal positions to effectuate two elongated chambers, and enable reciprocal movement of the plunger shaft in the syringe barrel to two, distinct, positions that are associated with the two filling steps, as described herein, for example in connection with the drawings. In other embodiments, the length of the plunger shaft receiving member may be variable (e.g., telescopically) to effectuate the two, distinct, positions. In other embodiments, the syringe liquid adjusting device may include two plunger shaft receiving members (for example two cylinders including a blind hole), where each plunger shaft receiving member is designed to enable a different reciprocal movement of the plunger shaft in the syringe barrel.

A typical syringe is operated by a person pushing and pulling a flange of a plunger shaft of the syringe towards (or away from) a flange of the syringe barrel. A flange of a syringe barrel is sometimes called a “barrel flange”, a “barrel tab”, a “hand tab”, a “hand grip”, a “finger support” and so on and so forth, so the term “barrel flange”, as used herein, refers to any structure of, or built into, a syringe barrel that is, or can be, used to move the plunger shaft in the syringe barrel. The term “infusion tube”, as used herein, may also include connector(s) which are used to connect the infusion tube to a container (for example an infusion syringe) containing liquid (e.g., medicament), and to connect one infusion tube section to another tube section, as the case may be (for example in cases where an infusion line includes tube sections that are connected to one another, via connector(s), to form a longer, continuous, infusion tube).

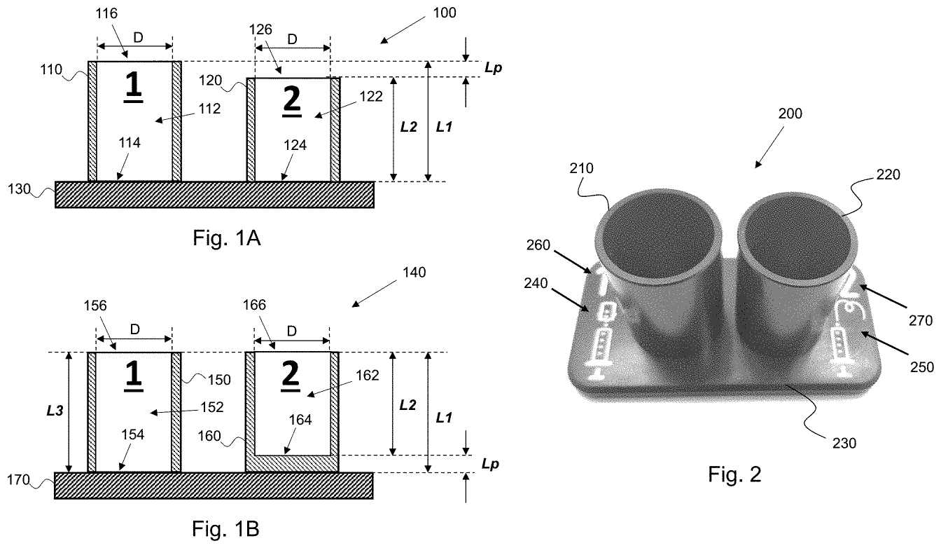

FIG. 1A shows a two-member syringe liquid adjusting device 100 for adjusting a liquid volume in a syringe barrel according to an example embodiment. Syringe liquid adjusting device 100 may include a first member 110 that includes a first elongated chamber 112 (an elongated blind hole). Elongated chamber 112 has a length L1 for receiving or accommodating (by being designed, for example sized and shaped, to do so) a partial length L1 of a plunger shaft of (moveable in) the syringe barrel. The value of L1 may correspond to a longitudinal position of the plunger shaft (relative to the barrel) where an initial liquid volume contained in the barrel is reduced to a total liquid volume that includes an infusion liquid volume for delivery to a subject. (An amount of liquid that fills up a syringe barrel is referred to herein as the “initial liquid volume”.)

First member 110 may include a proximal base 114 to abut (and apply a counteracting force to) the plunger flange of the plunger shaft to enable relative movement between the plunger shaft and the syringe barrel. First member 110 may also include a distal opening 116 opposite proximal base 114, to receive the plunger flange and the plunger shaft. Distal opening 116 may be configured to abut the barrel flange (and inhibit further movement of the barrel flange towards proximal base 114) for reducing the liquid volume in the syringe barrel from the initial volume to the total liquid volume.

Syringe liquid adjusting device 100 may also include a second member 120 that includes a second elongated chamber 122 (an elongated blind hole). Elongated chamber 122 has a length L2 for receiving or accommodating (by being designed, for example sized and shaped, to do so) a partial length L2 of the syringe barrel, where L2<L1. (Syringe liquid adjusting device 100 is a ‘two-member’ device, where each member includes a respective elongated chamber.)

The total liquid volume contained in the syringe barrel may additionally include a priming liquid volume for priming an infusion tube when the infusion tube is connected to the syringe, and the value of L2 may be selected to enable an additional relative movement between the plunger shaft and the syringe barrel in order to prime the infusion tube with the priming liquid volume when the liquid volume in the syringe barrel is reduced from the total liquid volume to the infusion liquid volume. From a slightly different point of view it may be said that the value of L2 may be selected such that an additional relative movement between the plunger shaft and the syringe barrel reduces the liquid volume in the syringe barrel from the total liquid volume to the infusion liquid volume while priming the infusion tube with the priming liquid volume. (The length difference Lp=L1−L2 is referred to herein as a “priming length difference”, as this length difference corresponds to expelling a liquid volume (the priming liquid volume) from a syringe barrel that is just enough to prime an infusion tube that is connected to the syringe. In some embodiments, though, the length difference Lp may provide also for some liquid volume margin that enables, for example, to ensure completeness of the priming process by expelling a few liquid droplets out of the infusion tube.)

Second member 120 may include a proximal base 124 to abut (and apply a counteracting force to) the plunger flange of the plunger shaft to enable relative movement between the plunger shaft and the syringe barrel. Second member 120 may also include a distal opening 126 opposite proximal base 124, to receive the plunger flange and the plunger shaft. Distal opening 126 may be configured to abut the barrel flange (and inhibit further movement of the barrel flange towards proximal base 124) for reducing the liquid volume in the syringe barrel from the total liquid volume to the infusion liquid volume while priming the infusion tube with the priming liquid volume.

The value of L1 is the distance between proximal base 114 and distal opening 116 of first member 110, and length L2 is the distance between proximal base 124 and distal opening 126. The distance difference Lp (Lp=L1−L2), as described herein, corresponds to the exact amount of liquid that is required to completely fill out the infusion tube when the infusion tube is connected to the syringe, and just before the infusion liquid volume is administered to the subject via the infusion tube. (The value of Lp can be chosen to suit any type of infusion tube.) In other words, Lp is a distance that the syringe barrel has to travel, relative to the plunger shaft (or plunger head), from a position corresponding to distance L1 to position corresponding to distance L2 in order to expel, from the syringe barrel, the exact liquid volume (optionally plus some margin) that is required to completely fill the infusion tube.

First member 110 and second member 120 may be formed as two separate objects where each object includes an elongated chamber, as illustrated in FIGS. 1A-1B. In other embodiments, the two members (110, 120) may be formed as one object that includes (encloses) the two elongated chambers.

In some embodiments, the first member and the second member have a different length, and each elongated chamber has a length that is equal to the length of the respective member. Referring to FIG. 1A, first member 110 and second member 120 have, respectively, a length equal to L1 and L2 (L1>L2), and each of elongated chamber 112 and elongated chamber 122 has a length equal to the respective member. In other embodiments, the first and second members have a same length, and the elongated chambers have a different length, as illustrated in FIG. 1B, which is described below.

FIG. 1B shows a two-member (dual-purpose) syringe liquid adjusting device 140 for adjusting a liquid volume in a syringe barrel according to another example embodiment. Syringe liquid adjusting device 140 includes a first member 150 that includes an elongated chamber 152 (a blind hole). Elongated chamber 152 is designed to receive (accommodate) a length, L1, of a plunger shaft of (moveable in) the syringe barrel. The value of L1 corresponds to a position of the plunger shaft, relative to the barrel, where an initial liquid volume contained in the syringe barrel is, or reduced to, a total liquid volume that includes an infusion liquid volume for delivery to a subject, and, optionally (e.g., in some embodiments), also a priming liquid volume for priming an infusion tube.

Syringe liquid adjusting device 140 may also include a second member 160 that includes an elongated chamber 162 (a blind hole). Elongated chamber 162 is designed to receive (accommodate) a length, L2, of a plunger shaft of (moveable in) the syringe barrel. The value of L2 corresponds to a position of the plunger shaft (relative to the barrel) where the total liquid volume contained in the syringe barrel is, or reduced to, the infusion liquid volume for delivery to a subject, and, in some embodiments, after having primed the infusion tube with a priming liquid volume. First member 150 and second member 160 can have the same length L1, and first elongated chamber 152 and second elongated chamber 162 have, respectively, a length equal to L1 and L2, where L1>L2.

A first member and ‘its’ elongated chamber may have the same length, as demonstrated by first members 110, 120 and 150 (see FIGS. 1A-1B) where first member 110 and elongated chamber 112 have the same length (e.g., L1), first member 120 and elongated chamber 122 have the same length (e.g., L2), and so on. One member of the two members, or both members, may include an elongated chamber whose length is shorter than the length of the respective member. For example (referring to FIG. 1B), elongated chamber 162 has a length L2 which is shorter than length L1 of member 160 (elongated chamber 162 is ‘shorter’ than elongated member 160). The first member and the second member may have a same length, but their elongated chambers may differ in length. For example, first member 150 and second member 160 may have a same length L3, but their elongated chambers may, respectively, have lengths L1 and L2. In the example of FIG. 1B, the length of member 150 is identical to the length of elongated chamber 152 (e.g., L3=L1), but this is not necessarily so because elongated chamber 152 may be shorter than first member 150; that is, L1 may be less (shorter) than L3 (L3>L1). In other words, elongated member 150 may have a similar structure as member 160.

The value of L1 is selected such that pushing the syringe barrel (by using the barrel flange or finger gripping tabs) against the plunger shaft when the plunger shaft resides in the first elongated chamber; e.g., in elongated chamber 110 (per the embodiment of FIG. 1A) or in elongated chamber 150 (per the embodiment of FIG. 1B), would result in the syringe barrel containing the total liquid volume. The value of L2 is selected such that further pushing the syringe barrel against the plunger shaft when the plunger resides in the second elongated chamber; e.g., elongated chamber 120 (per the embodiment of FIG. 1A) or in elongated chamber 160 (per the embodiment of FIG. 1B), would result in the priming of the infusion tube with the priming liquid volume from the syringe barrel, and leaving in the syringe barrel only the exact infusion liquid volume that is intended for delivery to the subject.

Each of the first member and the second member of a syringe liquid adjusting device may have a transverse cross-sectional area that is annular (e.g., that is close shaped) or non-annular (e.g., that is open shaped). Regardless of the exact shape of the transverse cross-sectional area of a member of a syringe liquid adjusting device, the member may be configured to receive, through its distal opening, the plunger's flange and shaft but not the barrel itself, as the opening of the member of a syringe liquid adjusting device may be made smaller than the barrel flange. For example, each opening of openings 116, 126, 156 and 166 may be sized and shaped (e.g., it may have an inner diameter D) to enable the opening to receive the plunger's flange and shaft but not the barrel flange.

The syringe liquid adjusting device may have a base member, which may be flat, and the first member and the second member of the syringe liquid adjusting device may outwardly protrude from the base member. Referring to FIGS. 1A-1B, syringe liquid adjusting device 100 may include a base member 130, which may be flat, from which first member 110 and second member 120 protrude outwardly, and syringe liquid adjusting device 140 may include a base member 170, which may be flat, from which first member 150 and second member 160 may protrude outwardly. Each of the first member and the second member may protrude perpendicularly from the respective base member. Bases 114 and 124, which are, respectively, the bases of members 110 and 120, may be part of (e.g., they may be continuation of) or attached to base member 130, and bases 154 and 164, which are, respectively, the bases of members 150 and 160, may be part of (e.g., they may be continuation of) or attached to base member 170.

In some embodiments, the functionalities of members 150 and 160 (FIG. 1B) may be combined, or implemented, in a single member, for example in member 150, whose overall length is, for example, L1, and the length L2 may be applied to the one member (e.g., member 150) by incorporating, into the one member, a removably insertable ‘inlay’ whose function is the same as the function of base 164 of member 160; namely, a ‘length reducer’ that ‘reduces’ the plunger shaft length accommodated by the single member from L1 to L2, or the plunger shaft length external to, or outside, the syringe barrel). That is, a structure element that functions in a same way as base 164 of member 160 may be designed to be insertable into, and removeable from, a ‘bottom’ (proximal) portion of member 150 (for example) in order to enable member 150 to function also as member 160, though at a different stage of the syringe liquid volume adjustment process. The removably insertable inlay may be implemented, for example, as a disc or ‘tab’ with a width that is equal to Lp. In such embodiments, before the inlay is inserted into member 150 this member can function as member 150 of FIG. 1B, and after the inlay is inserted into member 150 (while the inlay is inside member 150) the inlay, functioning like base 164, provides a raised base surface to member 150 to enable member 150 to function in the same way as member 160.

The annular shape of a member's transverse cross-sectional area may be, for example, circular, oval, ellipse and polygon, to name a few example shapes. The non-annular shape of the member's transverse cross-sectional area may generally be, for example, “U”-shaped. Each of members 110, 120, 150 and 160 may be made of, or include, at least one of a plastic material and a non-plastic material (e.g., metal). (Other materials may be used instead of, or jointly with, plastic and/or metal.)

FIG. 2 depicts a two-member syringe liquid adjusting device 200 for adjusting a liquid volume in a syringe barrel according to an example embodiment. Syringe liquid adjusting device 200 includes a first member 210, a second member 220 and a base 230, which may be flat. The two members (210, 220) protrude outwardly from base 230. Also shown in FIG. 2 are graphic user directives 240, 250, 260 and 270 that may be added to syringe liquid adjusting device 200, for example by engraving them on base 230 or by using other methods.

User directives 240 and 250 are for making it easier for a user (e.g., a patient) to decide which preparatory steps are associated with member 210 and which preparatory steps are associated with member 220. That is, before the user uses the first member (member 210), the user has to fill the syringe barrel with infusion liquid (e.g., medicament) from a vial, as user directive 240 directs. Then, the user is to insert the plunger's flange and shaft into this member and, using the member, expel excess liquid from the syringe barrel, for example back into the vial, in order to leave in the syringe barrel only the total liquid volume, which, as described herein, is the liquid volume required for both infusion and priming. (In some embodiments, the total liquid volume includes the infusion liquid volume but not a priming volume.) Likewise, before the user uses the second member (member 220), the user has to connect the infusion tube to the syringe's output port, as user directive 250 directs, and only then the user should insert the plunger's flange and shaft into this member, to thereby expel the priming liquid from the syringe barrel (to prime the infusion tube), while leaving in the syringe only the liquid volume required for the infusion.

User directives 260 and 270 are for indicating to a user (e.g., a patient) which member should be used first and which member should be used next. In this example member 210 should be used first so it is designated as number “1”, and member 220 should be used next so it is designated as number “2”. (Other types of user directives may be used as alternatives or in addition to the example user directives described/mention herein.)

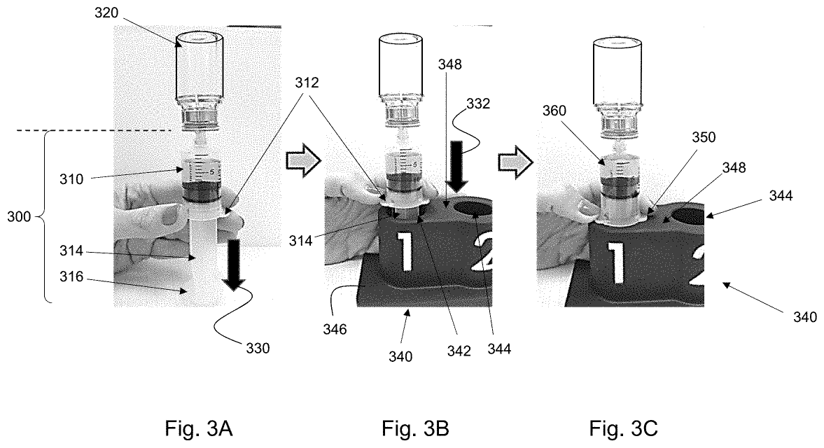

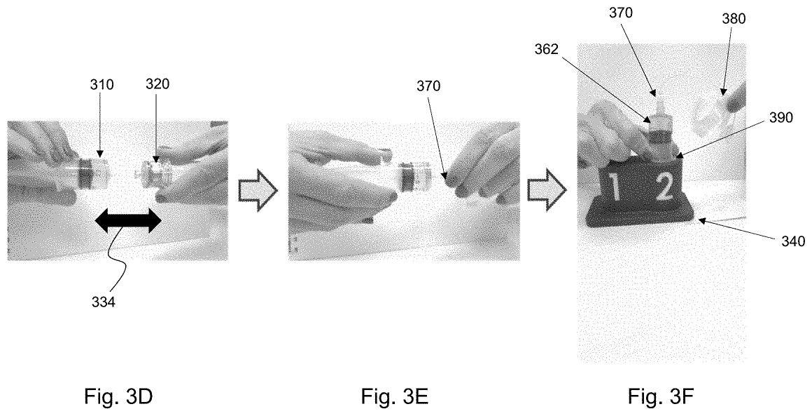

FIGS. 3A-3F depict various steps of a method of adjusting a liquid volume in a syringe barrel according to an example embodiment. Referring to FIG. 3A, a syringe 300 includes a syringe barrel 310, a barrel flange 312, a plunger shaft 314 and a plunger flange 316. In preparation of the syringe 300, for example, for infusing its liquid content (e.g., medicament), a user of syringe 300 (e.g., a patient, a caregiver, etc.) fills up syringe 300 by pulling plunger flange 316 away from syringe barrel 310, in direction 330, to thereby draw liquid from a liquid source (e.g., vial 320) into syringe barrel 310. (It is assumed that filled-up syringe barrel 310 contains a liquid volume that contains at least the liquid volume required for infusion plus a liquid volume that is required for priming an infusion tube. In other embodiments, filled-up syringe barrel 310 may contain a liquid volume that contains at least the liquid volume required for infusion, but not a priming liquid volume.)

FIG. 3B also depicts a ‘single-object’ syringe liquid adjusting device 340 that includes two elongated chambers (342, 344). (The two, separate, members of the syringe liquid adjusting device of FIGS. 1A-1B and FIG. 2 can be replaced by one object (the object making up syringe liquid adjusting device 340) that combines the two-member device and, as in the two-member syringe liquid adjustment device, includes two elongated chambers, as demonstrated by FIG. 3B, at 342 and 344. The two functions that are performed, for example, by members 110 and 120 (or by members 150 and 160); namely, adjusting the amount of liquid in a syringe barrel to the total liquid volume and priming an infusion tube, can be performed by using a single object that may be identical or similar to the object making up syringe liquid adjusting device 340).

After the user uses vial 320 to fill up syringe barrel 310 with the initial liquid volume, the user may insert the plunger shaft (314) and flange (316) into first elongated chamber 342 of syringe liquid adjusting device 340. (First elongated chamber 342 of syringe liquid adjusting device 340 is designated as number “1”, and its function is similar to the function of other first elongated chambers described herein, for example in connection with FIGS. 1A-1B and FIG. 2.) Then, the user may push, or slide, barrel flange 312 onto plunger shaft 314 and against base 346 of syringe liquid adjusting device 340, in direction 332, to transfer an excess liquid volume (if there is any) back into vial 320.

When barrel flange 312 abuts distal surface 348 of syringe liquid adjusting device 340 (the abutment is shown in FIG. 3C at 350), the remaining liquid volume (360) in syringe barrel 310 is the total liquid volume, which is an amount of liquid that is the sum of the infusion liquid volume (the liquid volume required for infusion) and the priming liquid volume (the liquid volume required for priming an infusion tube).

FIG. 3D and FIG. 3E respectively depict a step in which the user disconnects (334) vial 320 from the output/input port of syringe barrel 310 and, instead, connects an infusion tube to the syringe. (An end portion of the infusion tube is shown in FIG. 3E at 370. The infusion tube is shown in FIG. 3F at 380.)

Referencing FIG. 3F, when the infusion tube is connected to the syringe (the syringe may be a Luer lock syringe), the user may insert the plunger shaft (314) and flange (316) into second elongated chamber 344 of syringe liquid adjusting device 340. (Second elongated chamber 344 of syringe liquid adjusting device 340 is designated as number “2”, and its function is similar to the function of other second elongated chambers described herein, for example in connection with FIGS. 1A-1B and FIG. 2.) Then, the user may push, or slide, barrel flange 312 further onto plunger shaft 314 and against base 346, which may be flat, of syringe liquid adjusting device 340 until barrel flange 312 abuts distal surface 348 of syringe liquid adjusting device 340 (the abutment is shown in FIG. 3F at 390).

Moving barrel flange 312 towards the ‘abutment position’ on syringe liquid adjusting device 340 expels the priming liquid volume from syringe barrel 310 into the infusion tube, to thereby prime the infusion tube. When barrel flange 312 abuts distal surface 348 of syringe liquid adjusting device 340 (per FIG. 3F, abutment position 390), the infusion tube is completely primed and the remaining liquid volume (362) in syringe barrel 310 is the infusion liquid volume, which is the amount of liquid required for infusion. At this stage (after priming of the infusion tube is completed), the user may take the syringe out of syringe liquid adjusting device 340 and operate the syringe, with the infusion tube connected thereto, to perform the infusion process itself

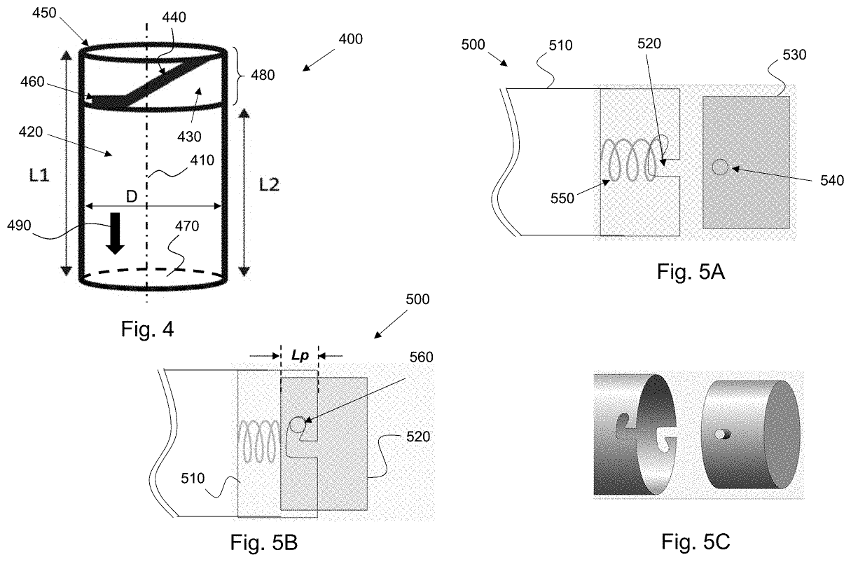

FIG. 4 schematically illustrates a single-member, dual function, syringe liquid adjusting device 400 according to another example embodiment. The term “dual function” refers herein to a member that, by having a variable length, provides the two elongated chambers corresponding to L1 and L2, as described herein, where each elongated chamber is associated with (enables performing) one of the two functions described herein. Namely, one elongated chamber enables reducing a liquid volume in a syringe barrel from the initial liquid volume to the total liquid volume, and the other elongated chamber enables reducing the liquid volume in the syringe barrel from the total liquid volume to the infusion liquid volume.

The two functions that are described herein in connection with the two-member device (e.g., using the first member to fill up a syringe barrel with a liquid volume including the infusion liquid volume and, optionally, also the priming liquid volume, and, using the second member, to prime the infusion tube while leaving in the syringe barrel the exact amount of liquid that required for the infused) can be implemented, according to the embodiment of FIG. 4, by syringe liquid adjusting device 400, which may be regarded as a ‘one-member’, or ‘single-member’, syringe liquid adjusting device. (The “one member” making up device 400 is also referred to as the “first member”, though device 400 includes only this member; that is, in this case there is no “second member”.)

Referring to FIG. 4, syringe liquid adjusting device 400 has an axis 410 and includes two functionally distinct axial portions (portions 420, 430) along axis 410, that are used as the first chamber and second chamber whose function is described throughout the description.

Syringe liquid adjusting device 400 also includes a ‘guide’ 440 that enables a barrel, or a barrel flange, to move (e.g., linearly, or through rotation thereof) between two distinct axial positions 450 and 460 in or on syringe liquid adjusting device 400. Guide 440 may be, include or be part of a helical slot, a groove, a recess or a thread. Syringe liquid adjusting device 400, by using guide 440, enables performing the two functions that are described herein by defining the two axial positions 450 and 460, where each axial position provides for a different position of plunger shaft 314 (see FIG. 3A) relative to syringe barrel 310 (see FIG. 3A). For example, axial position 450 provides for a position of plunger shaft 314 (relative to the syringe barrel) that corresponds to length L1, and axial position 460 provides for a position of plunger shaft 314 (relative to the syringe barrel) that corresponds to length L2. Therefore, even though one-member device 400 includes one member, it provides two elongated chambers that function in a similar way as elongated chambers 112 and 122 (FIG. 1A) and elongated chambers 152 and 162 (FIG. 1B). Namely, one-member device 400 effectuates two, distinct, elongated chambers that enable reciprocal movement of the plunger shaft in the syringe barrel from a position corresponding to distance L1 to a position corresponding to distance L2, and vice versa. (Axial portion 420 serves as the first elongated chamber, and axial portions 420 and 430 jointly (in combination) make the second elongated chamber.

As described herein, the total liquid volume that is contained in the syringe barrel after the liquid volume in the barrel is reduced from an initial liquid volume by using the first member (or first elongated chamber) may include, in some embodiments, only a liquid volume that is equal to the infusion liquid volume. However, in other embodiments, the total liquid volume additionally includes a priming liquid volume for priming an infusion tube.

Referring again to FIG. 4, first member 400 (one-member device) may further include a counteracting base 470 and an opening portion 480 opposite counteracting base 470. Opening portion 480, through guide 440, enables operating first member 400 in two operational states that, respectively, correspond to axial position 450 and to axial position 460 in first member 400.

Opening portion 480, which is configured (e.g., shaped and sized, has a structure) to enable receiving there through the length L1 of a plunger shaft, provides for two operational states that are related to two, axially spaced apart, positions. In a first operational state, the barrel flange is at (abuts) axial position 450 so that the distance between a flange of the syringe barrel and a flange of the plunger shaft is reduced to L1 to reduce the liquid volume in the barrel from the initial liquid volume to the total liquid volume including infusion liquid volume and priming liquid volume. In the second operational state, the barrel flange is at (and, in some embodiments, may be reversibly locked/lockable in or latched to) axial position 460 so that the distance between the barrel flange and the plunger flange is reduced from L1 to a value L2 to prime the infusion tube with the priming liquid volume contained in the barrel while reducing the liquid volume in the barrel from the total liquid volume to the infusion liquid volume. (Axial position 450 may be referred to as an ‘abutment position’. In some embodiments, the flange of the syringe barrel may be locked in, or to, axial position 460, and for such embodiments, axial position 460 may be referred to as a ‘locking position’. The length difference, Lp, between these two positions (450, 460) facilitates the priming step described herein. The length L1 may be defined, for example, as a distance between a barrel flange, when it is in the abutment position, and proximal counteracting base 470 of member 400.)

In some embodiments, the two operational states are: (i) an abutment state corresponding to axial abutment position 450, and (ii) a locking state corresponding to axial locking position 460 in which the opening portion locks the barrel flange in the locking position, which is a different location than the location associated with the abutment position. Referring to the abutment state, when plunger shaft 314 is inserted (e.g., by a user) into first member 400 (in direction 490) and the barrel flange, or the syringe barrel, is moved in direction 490 (towards counteracting base 470) to abutment position 450, the distance between the barrel flange and counteracting base 470 is reduced to L1 while expelling excess liquid volume out of the syringe barrel. (The value of L1 corresponds to the syringe barrel containing only the total liquid volume.) Referring to the locking state, when the barrel or barrel flange is further moved (in direction 490) from abutment position 450 to locking position 460, the distance between the barrel flange and counteracting base 470 is reduced from L1 to L2 to prime the infusion tube with the priming liquid volume while reducing the liquid volume in the syringe barrel from the total liquid volume to the infusion liquid volume. (The length difference Lp=L1−L2 corresponds to a syringe liquid required to prime the infusion tube and is calculated accordingly.)

Opening portion 480 includes guide 440 that the barrel flange, or syringe barrel, follows when it is moved from abutment position 450 to locking position 460, and vice versa (e.g., when the barrel flange, or syringe barrel, is released from the locking position and removed from first member 400. Opening portion 480 may additionally include a locking mechanism to detachably lock (secure) the syringe barrel (or the barrel flange) in locking position 460. Locking the syringe barrel (or the barrel flange) in locking position 460 has advantages; for example, this feature prevents reflux of liquid from the infusion tube back into the syringe while all air and/or liquid pressures, which may be built up in the syringe and/or infusion tube during the priming operation are equalized.

Portion 420 of first member 400 may be designed (e.g., sized and shaped, for example it may have an inner diameter D) to receive a plunger shaft (for example plunger shaft 314) but not the barrel flange. Portion 430 of first member 400 may be designed (e.g., sized and shaped) to receive a plunger shaft (for example plunger shaft 314) and the barrel flange when the barrel flange is at an ‘engaging orientation’ with respect to guide 440 in or of opening portion 480. (The barrel flange cannot travel the distance Lp (Lp=L1−L2), linearly or rotationally, inside opening portion 480 if the barrel flange is misoriented with respect to barrel flange guide 440, in which case the barrel flange cannot engage (be guided by) guide 440. Guide 440 may be a linear guide or a helical guide.)

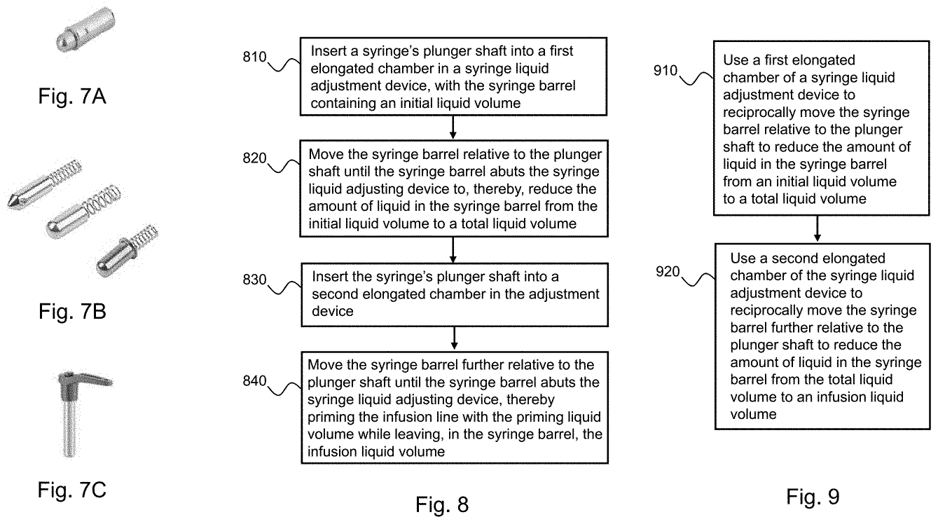

The locking mechanism may include a longitudinal position setting means that may be implemented as, or using, a spring plunger (an example spring plunger is shown in FIG. 7A), a positioning setting spring sleeve (an example spring sleeves are shown in FIG. 7B), a ball lock pin (an example ball lock pin is shown in FIG. 7C), or a similar device. (The longitudinal position setting means may be implemented using other devices, or a combination of devices.) The longitudinal position setting means (e.g., locking mechanism) may be mounted in (or be part of) member 400, and a matching (a ‘complementing’ or mating means) hole, slot, groove, notch, indentation or void may be formed on or in the syringe barrel and/or in the barrel flange. (The locking mechanism may be designed for locking conventional or non-conventional syringe barrels and barrel flanges.) The locking mechanism may be, include, or be part of a helix slot, a nut, a thread or a bayonet coupling receptacle or any other ‘twist and snap’ design, etc.

The locking mechanism may be implemented, for example, as a bayonet coupling mechanism such as the one shown in FIGS. 5A-5C. Bayonet coupling mechanism 500 includes a bayonet receptacle 510, which includes a guide 520, and a plug 530, which includes a bayonet pin 540. Bayonet receptacle 510 is engageable with plug 530 by means of guide 520 and bayonet pin 540. Bayonet receptacle 510 may include a spring 550 to apply a counteracting force on plug 530 when bayonet pin 540 is in place (560, FIG. 5A). FIG. 5A shows the two parts (bayonet receptacle 510 and plug 530) before the engagement, and FIG. 5B shows the two parts engaged. Opening portion 480 of first member 400 (FIG. 4) can be, include or designed in a similar way as bayonet receptacle 510, and the syringe barrel, or barrel flange, can be designed in a similar way as plug 530, so that bayonet receptacle 510 and the syringe barrel, or barrel flange, can be engaged in a similar way as bayonet receptacle 510 and plug 530. FIG. 5C depicts an example bayonet receptacle and plug.

After the infusion tube connected to the syringe barrel is primed with the priming liquid volume, the syringe barrel (or the barrel flange) may be released (e.g., detached, unscrewed, unplugged) from the locking mechanism and removed, first from locking position 460 and, then, altogether from first member 400. Then, the infusion tube may be attached to the person requiring infusion, and the infusion process may commence.

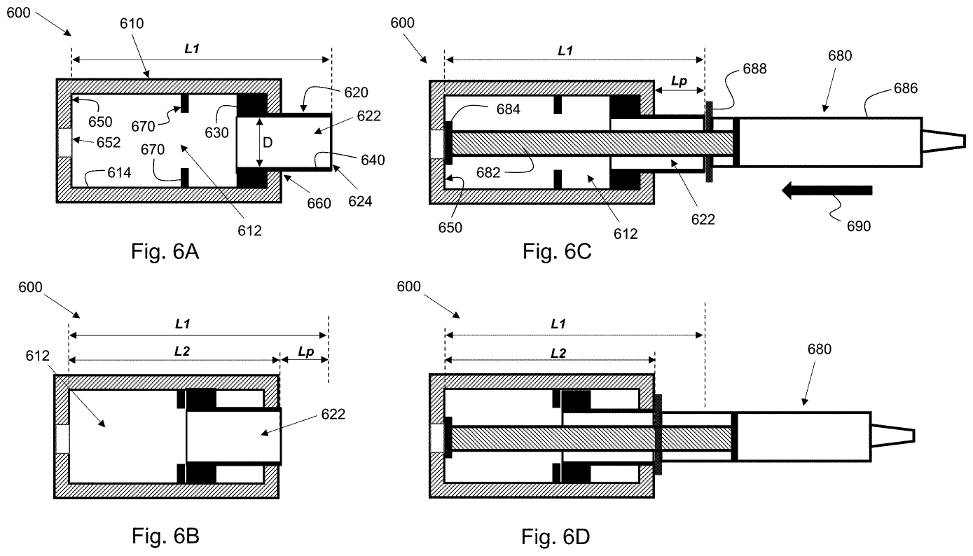

FIGS. 6A-6B show a cross-sectional view schematically illustrating a single-member, dual-function, syringe liquid adjusting device 600 (a telescopic device) according to another example embodiment. The two functions that are described herein in connection with the two-member device (e.g., see FIGS. 1A-1B). Namely, using the first member to fill up a syringe barrel with a liquid volume including the infusion liquid volume and, in some embodiments, also the priming liquid volume (reducing the liquid volume of a syringe barrel from the initial liquid volume to the total liquid volume), and, using the second member, to prime the infusion tube while leaving in the syringe barrel the exact amount of liquid that required for the infused (reducing the liquid volume of the syringe barrel from the total liquid volume to the infusion liquid volume) can be implemented, according to the embodiment of FIGS. 6A-6D, by single-member syringe liquid adjusting device 600. (Syringe liquid adjusting device 600 is also referred to as the “first member” as it includes only one member, yet it performs the two functions that are described herein, for example, in connection with the two-member devices and in connection with the single-member device of FIG. 4.)

First member 600 has a telescopic structure (member 600 is a telescopic device) that includes, among other things, an outer member 610 and an inner member 620. Outer member 610 may include a proximal base (a ‘counteracting’ base) 650 (which may include a through hole 652, for example to serve as a vent path) and a distal opening 660 opposite proximal base 650. Inner member 620 may include a ring-like base part 630 and a cylindrical body 640 that includes a through hole 622 (to implement an elongated ‘chamber’) and protrudes from ring-like base part 630 and through distal opening 660 of outer member 610. Elongated chamber 622 of inner member 620 and an elongated chamber 612 of outer member 610 are designed (e.g., sized and shaped) such that a user can easily insert a plunger flange and shaft (but not a barrel flange) through chamber 622 and into elongated chamber 612 of telescopic member 600.

Proximal base 650, by applying a counteracting force to shaft flange 684, enables the reciprocal movement of syringe barrel 686 relative to syringe shaft 682 (hence the term “counteracting base”).

Outer member 610 and inner member 620 are configured such that inner member 620 is concentrically and reciprocally moveable in outer member 610, optionally along one or more guiding tracks, in a longitudinal manner, between two, spaced apart, axial positions: (i) an ‘extended position’, in which inner member 620 concentrically extends out from outer member 610 to a first axial position (relative to outer member 610) that enables accommodating (by member 600) a length L1 of the plunger shaft, as shown in FIG. 6A, and, (ii) a ‘retracted position’, in which inner member 620 is concentrically retracted into outer member 610 to a second axial position, different than the first axial position, that enables accommodating (by member 600) a length L2 (L2<L1) of the plunger shaft, as shown in FIG. 6B.

A ledge 670 (e.g., a ‘seat’, lug, protrusion, etc.) may be formed inside, attached to or be an integral part of an inner wall 614 of outer member 610 at a location corresponding to the second axial position (L2), in order to prevent inner member 620 from moving passed that point. As FIGS. 6A-6B show, one-member device 600 provides an elongated chamber whose length is variable; namely, its length is changeable from length L1 (when member 600 is in the extended position), to length L2 (when member 600 is in the retracted position). The length difference Lp=L1−L2, which is mentioned throughout the specification and shown, for example, in FIGS. 1A-1B, FIG. 5B and FIG. 6B, corresponds to the liquid volume required to completely fill (i.e., prime) an infusion tube connected to the syringe.

First (telescopic) member 600 may also include a locking mechanism that may be configured for locking inner member 620 in, or to, outer member 610 in the extended position, or in the retracted position, or both in the extended and retracted positions. (The extended and retracted positions are two axially spaced apart and distinct positions. The locking mechanism is not shown in FIGS. 6A-6B.)

The locking mechanism may include a longitudinal position setting means that may be implemented as, or using, a spring plunger (an example spring plunger is shown in FIG. 7A), a positioning setting spring sleeve (an example spring sleeves are shown in FIG. 7B), a ball lock pin (an example ball lock pin is shown in FIG. 7C), or a similar device. (The longitudinal position setting means may be implemented using other devices, or a combination of devices.) The longitudinal position setting means may be mounted in (and be part of) one of the outer member (610) and the inner member (620), and a matching (a ‘complementing’, mating means) hole, slot, groove, notch, indentation or void may be formed on or in one of the other one of the outer member (610) and the inner member (620).

In some embodiments, two longitudinal position setting means may be used: a first one to lock inner member 620 in, or to, outer member 610 in the extended position, and a second one to lock inner member 620 in, or to, outer member 610 when inner member 620 is in the retracted position. In other embodiments, one longitudinal position setting means may be used to lock inner member 620 in, or to, outer member 610 when inner member 620 in in the extended position or in the retracted position.

The devices of FIGS. 7A-7C (and like or other devices) can be used for quick fastening, locking, adjusting and securing of inner member 620 in, or to, outer member 610. It is assumed that any person skilled in the art would be able to make and use the invention without undue experimentation, including properly fitting a locking mechanism to member 600. Therefore, additional information, for example regarding how to implement a locking mechanism in member 600, does not seem to be necessary.

FIGS. 6C-6D show a cross-sectional view of an example syringe 680 accommodated by one-member syringe liquid adjusting device 600 in the two, distinct, positions: (i) in the extended position of device 600 (per FIGS. 6A and 6C), and (ii) in the retracted position of device 600 (per FIGS. 6B and 6D). Referring to FIG. 6C, syringe 680 includes a plunger shaft 682, a plunger flange 684, a barrel 686 and a barrel flange 688. Initially, inner member 620 is extended; i.e., brought to the extended position by pulling it (partly), by a user, outer member 610. Then, syringe 680 is inserted (by the user) into the elongated chamber 612 of member 600, in direction 690, until plunger flange 684 abuts proximal base 650 of device 600, as shown in FIG. 6C (for example). Then, barrel 686 (or barrel flange 688) is pushed (e.g., by the user) against member 600, in direction 690, until barrel flange 688 abuts the distal end 624 of inner member 620. In this state, the elongated chamber provided by member 600 (e.g., chamber 612 and chamber 622 jointly form one, elongated, chamber) accommodate a length L1 of the plunger shaft.

Chambers 612 and 622 accommodate different portions of plunger shaft 682, but since inner member 620 partly resides in outer member 610, elongated chambers 612 and 622 partly overlap, with the overlapping portions accommodating a same portion of plunger shaft 682. The overlapping between chambers 612 and 622 is minimal when telescopic member 600 is in the extended position or state, and maximal when telescopic member 600 is in the retracted position or state. (The overlapping between members 610 and 620 changes between minimal and maximal overlapping during reciprocal movement of inner member 620 in outer member 610 between the extended position and the retracted position.

While barrel 686 is moved (by a user) towards abutment with inner member 620, an initial liquid volume (with which barrel 686 is prefilled by a user) is reduced to a total liquid volume that includes an infusion liquid volume and a priming liquid volume for priming an infusion tube. (The infusion tube is not shown in FIGS. 6C-6D). (At this stage syringe 680 may be removed from member 600, and an infusion tube may be connected to syringe 680 in preparation for the priming step and infusion.)

Referring to FIG. 6D, inner member 620 is retracted (brought to the member's retracted position by inserting it, by a user, into outer member 610). Then, syringe 680 may be inserted again into member 600, in direction 690, until plunger flange 684 abuts proximal base 650, as shown in FIG. 6D. Then, barrel 686 (or barrel flange 688) may be further pushed (by the user) against member 600, in direction 690, until barrel flange 688 abuts the distal end of inner member 620. In this state, the elongated chamber provided by member 600 (e.g., chamber 612 of outer member 610 and chamber 622 of inner member 620 that jointly form the elongated chamber) accommodates a length L2 of the plunger shaft, where L2<L1.

While barrel 686 is moved the second time towards abutment with inner member 620, the priming liquid volume (which is part of the total liquid volume contained in syringe barrel 686) is expelled (by the reciprocal movement of plunger shaft 682 in barrel 686) to prime the infusion tube. When the infusion tube priming process is completed, the only liquid volume that remains in syringe barrel 686 is the infusion liquid volume. The value of L2 is selected to enable an additional relative movement between plunger shaft 682 and syringe barrel 686 in order to prime the infusion tube with the priming liquid volume while reducing the liquid volume in syringe barrel 686 from the total liquid volume to the infusion liquid volume.

Syringe liquid adjusting device 600, by using telescopic members 610 and 620, enables performing the two functions that are described herein by defining the two axially spaced positions (extended position corresponding to length L1, and retracted position corresponding to length L2), where each axial position provides for a different position of plunger shaft 682 relative to syringe barrel 686. Therefore, even though member 600 is basically one object, it provides two elongated chambers (not at the same time) that function in a similar way as, for example, elongated chambers 112 and 122 (FIG. 1A) and elongated chambers 152 and 162 (FIG. 1B). Namely, member 600 provides for two, distinct, elongated chambers that enable reciprocal movement of plunger shaft 682 in syringe barrel 686 from a position corresponding to distance L1 (e.g., from the extended position) to a position corresponding to distance L2 (e.g., to the retracted position), and vice versa.

Each of the first member and the second member of the two-member embodiments, and the first member of the one-member embodiments may include a plastic material, or non-plastic material (e.g., metal), or a combination of plastic and non-plastic materials.

Each of the first member and the second member of the two-member embodiments may have an internal diameter, D, whose value may be selected from the range of 10 mm-30 mm. For example, the value of D may be 28.50 mm.

The values of L1 and L2 may be calculated based on, or using, one or more of the following parameters: (1) a maximum volume of liquid that the syringe barrel can contain, (2) a volume of liquid that is required to fill up the entire infusion tube; (3) a volume of liquid that is intended to be infused to the subject; (4) a size of the syringe barrel; (5) total length and inner diameter of the infusion tube; (6) a cross-sectional area of the syringe barrel; and (7) an internal diameter of the barrel. The length difference, Lp (where Lp=L1−L2), can be calculated based on (it corresponds to) the priming liquid volume that is required to completely fill up the entire infusion tube. The value of Lp may be selected, for example, from the range of 0.36 mm-3.0 mm. For example, the value of Lp may be selected from the group consisting of, for example, 0.85 mm, 0.4 mm and 0.36 mm. The value of L1 may be selected, from example, from the range 50 mm-54 mm, and the value of L2 may be selected, for example, from the range 49 mm-53 mm. By way of example, the value of L1 may be selected from the group consisting of 52.65 mm, 52.16 mm and 52.2 mm, and the value of L2 may be selected from the group consisting of 51.80 mm and 52.29 mm. In some embodiments, the values of L1 and L2 may be selected so as to accommodate for (to suit or ‘serve’) a variety (e.g., a selection) of conventional syringes and non-conventional syringes.

Referring again to FIG. 4, member 400 has a fixed length, L1, including two, fixed, positions (abutment position 450 and locking position 460) that correspond, facilitate or provide the two elongated chambers. Referring again to FIGS. 6A-6D, member 600 has a variable length that varies between the extended position length, L1, and the retracted position length, L2, that are respectively corresponding to the first elongated chamber and the second elongated chamber. (L1, L2 and Lp may be adjustable according to circumstances, for example in order to accommodate for a selection of types of syringes and/or infusion tubes.)

The distance between the barrel flange and the plunger flange (or, equivalently, counteracting base) may initially have an initial value that is greater than L1, so that the barrel initially contains the initial liquid volume, and the initial value greater than L1 can be reduced to L1, which, as described herein, corresponds to the barrel containing the total liquid volume.

FIG. 8 shows a method of operating a syringe liquid adjustment device identical or similar to syringe liquid adjustment devices 100, 140, 200, 340, 400 and 600 to adjust a liquid volume in a barrel of a syringe according to example embodiments. For example, the initial liquid volume is equal to or greater than a total liquid volume that, in these embodiments, is equal to the infusion liquid volume and the priming liquid volume combined. In these embodiments, two elongated chambers are required to perform the two steps described herein: (1) reducing the liquid volume in the syringe barrel from the initial liquid volume to the total liquid volume, and (2) priming the infusion tube. The method shown in FIG. 7 refers to these embodiments.

At step 810 a user (e.g., a patient, a caregiver, medical staff, etc.) inserts a plunger shaft (e.g., plunger shafts 314, 682) of the syringe (e.g., syringes 300, 680) into a first elongated chamber in a syringe liquid adjustment device. The first elongated chamber (e.g., first elongated chambers 112, 152, combined portions 420 and 430, combined portions 612 and 622 in the extended state, etc.) may be formed in, or implemented by, a first member (e.g., first members 110, 150, 400, 600) of the syringe liquid adjustment device. The first elongated chamber may be configured to receive a length, L1, of, the plunger shaft but not the syringe barrel, and the syringe barrel may pre-filled, for example at this stage, with an initial liquid volume. Step 810 may include a step of, or including, prefilling the syringe barrel with the initial liquid volume.

At step 820, with the plunger shaft of the syringe inserted into the first elongated chamber, the user moves, or slides, the syringe barrel (e.g., by using a barrel flange of the syringe barrel) against, or relative to, the plunger shaft until the syringe barrel is brought to abutment position in which the syringe barrel abuts the syringe liquid adjustment device (e.g., devices 100, 140, 200, 400, 600), to thereby reduce the initial liquid volume to a total liquid volume which includes an infusion liquid volume for delivery to a subject and a priming liquid volume to prime an infusion tube connectable to the syringe.

At step 830, the user inserts the plunger shaft into a second elongated chamber of the syringe liquid adjustment device. The second elongated chamber (e.g., elongated chambers 122, 162, portion 420, combined elongated chambers 612 and 622 in the retracted state; i.e., having a shorter overall length relative to their overall length in the extended state) may be formed in, or implemented by, a second, separate (second), member, or/by the same (e.g., first) member. (The first and second elongated chambers may be formed in or implemented by the same member.) Depending on their stage of use, or according to context, members 400 and 600 are sometimes referred to (e.g., in the description relating to their function) as the “first member” and sometimes as the “second member”, even though a same member is involved.) The second elongated chamber is configured to receive a length, L2, of the plunger shaft but not the syringe barrel. (L2<L1).

At step 840, the user moves, or slides, the syringe barrel further against, or relative to, the plunger shaft until the syringe barrel abuts the syringe liquid adjusting device, thereby priming the infusion tube with the priming liquid volume while leaving, in the syringe barrel, the infusion liquid volume in preparation for the next ‘phase’/step, which is patient infusion.

As described herein, in some embodiments a syringe liquid adjusting device may include only one member (e.g., member 400 in FIG. 4, member 600 in FIGS. 6A-6D) that functions in a similar way as (and thus can be used instead of) the first and second members of the two-member syringe liquid adjustment devices, in a sense that the single-member syringe liquid adjusting device provides an elongated chamber whose length is variable between two, distinct, lengths that can also be provided by the two-member devices.

Step 820 may include a step in which the user removes the syringe, including the plunger shaft, from the first elongated chamber, and connects an infusion tube to the syringe. (‘ . . . remove the syringe’ primarily refers to the liquid adjustment device embodiments where the syringe needs to be removed from one elongated chamber and inserted into another, separate, elongated chamber. However, in the one-member embodiments, the method of using the first elongated chamber and the second elongated chamber can be performed while the plunger shaft of the syringe remains in the ‘first member’ (e.g., in the one-member device), meaning that using a single-member renders the removal of the syringe unnecessary.)

In some embodiments, the total liquid volume is equal to the infusion liquid volume, and, in these embodiments, one elongated chamber is required to perform the one step described herein; namely, reducing the liquid volume in the syringe barrel from the initial liquid volume to the total liquid volume which, in this case, is equal to the infusion liquid volume.

FIG. 9 shows a method of operating a syringe liquid adjustment device identical or similar to syringe liquid adjustment device 100, 140, 200, 340, 400 and 600 to adjust a liquid volume in a barrel of a syringe according to example embodiments. At step 910, a user uses a first elongated chamber of a syringe liquid adjustment device to reciprocally move the syringe barrel relative to the plunger shaft of the syringe to reduce the amount of liquid in the syringe barrel from an initial liquid volume to a total liquid volume that includes an infusion liquid volume and a priming liquid volume. At step 920, the user uses a second elongated chamber of the syringe liquid adjustment device to reciprocally move the syringe barrel further relative to the plunger shaft to reduce the amount of liquid in the syringe barrel from the total liquid volume to an infusion liquid volume while priming an infusion tube with the priming liquid volume.

The articles “a” and “an” are used herein to refer to one or to more than one (e.g., to at least one) of the grammatical object of the article, depending on the context. By way of example, depending on the context, “an element” can mean one element or more than one element. The term “including” is used herein to mean, and is used interchangeably with, the phrase “including but not limited to”. The terms “or” and “and” are used herein to mean, and are used interchangeably with, the term “and/or,” unless context clearly indicates otherwise. The term “such as” is used herein to mean, and is used interchangeably, with the phrase “such as but not limited to”.

Having thus described exemplary embodiments of the invention, it will be apparent to those skilled in the art that modifications of the disclosed embodiments will be within the scope of the invention. Alternative embodiments may, accordingly, include functionally equivalent objects/articles. Features of certain embodiments may be used with other embodiments shown herein. The present disclosure is relevant to (e.g., it may be used with or for) various types of syringes, drugs, liquids in general, infusion tubes/sets/lines, liquid dispensing devices, and the like. Hence the scope of the claims that follow is not limited by the disclosure herein.

1. A device for adjusting a liquid volume in a barrel of a syringe, the device comprising:
a first member, said first member comprising a first elongated chamber having a length L1 for receiving a length L1 of a plunger shaft of the syringe;
wherein the value of L1 corresponds to a position of the plunger shaft, relative to the barrel, where a liquid volume contained in the barrel is a total liquid volume including an infusion liquid volume intended for patient infusion.

2. The device as in claim 1, wherein the total liquid volume additionally includes a priming liquid volume for priming an infusion tube, the first member further comprising:
an opening portion, to receive there through the length L1 of the plunger shaft, wherein the opening portion provides for two operational states related to two axial positions:
(i) a first state in which the distance between a flange of the barrel and a flange of plunger shaft is reduceable to L1, and
(ii) a second state in which the distance between the barrel flange and the plunger flange is reduceable from L1 to a value L2 to prime the infusion tube with the priming liquid volume contained in the barrel while reducing the liquid volume in the barrel from the total liquid volume to the infusion liquid volume.

3. The device as in claim 2, wherein a first axial position of the two axial positions is an abutment position in which the barrel flange abuts the first member, and a second axial position of the two axial positions is a locking position in which the barrel flange is reversibly lockable.

4. The device as in claim 3, wherein the opening portion comprises a locking structure to lock the barrel flange in the locking position.

5. The device as in claim 4, wherein the locking structure comprises a helix slot, a nut, a thread or a bayonet coupling receptacle.

6. The device as in claim 2, wherein the first member is a telescopic structure comprising:
an outer member; and
an inner member concentrically residing in said outer member and configured for reciprocal movement in the outer member between two positions:
(i) an extended position in which the inner member extends from the outer member to enable accommodating the length L1 of the plunger shaft, and
(ii) a retracted position in which the inner member is retracted into the outer member to enable accommodating a length L2 of the plunger shaft, where L2<L1,
wherein the total liquid volume further includes a priming liquid volume for priming an infusion tube, and wherein the value of L2 is selected to enable an additional relative movement between the plunger shaft and the barrel in order to prime the infusion tube with the priming liquid volume while reducing the liquid volume in the barrel from the total liquid volume to the infusion liquid volume.

7. The device as in claim 6, wherein the first member further comprises a locking mechanism to reversibly lock the inner member in, or to, the outer member in the extended position, or in the retracted position, or both in the extended and retracted positions.

8. The device as in claim 7, wherein locking mechanism comprises a ball lock pin, a positioning setting spring sleeve and a spring plunger on or in one of the outer member and the inner member, and a matching hole, slot, groove, notch, indentation or void on or in one of the other one of the outer elongated hollow member and the inner elongated hollow member.

9. The device as in claim 2, wherein the total liquid volume additionally includes a priming liquid volume for priming an infusion tube, and the first member further comprising:
a removably insertable inlay, said insertable inlay providing two operational states:
(i) a first operational state in which the distance between a flange of the barrel and a flange of the plunger shaft is reduceable to L1, and
(ii) a second operational state in which the distance between the barrel flange and the plunger flange is reduceable from L1 to a value L2 to prime the infusion tube with the priming liquid volume contained in the barrel while reducing the liquid volume in the barrel from the total liquid volume to the infusion liquid volume.

10. The device as in claim 1, and further comprising:
a second member, said second member comprises a second elongated chamber configured to receive a second length, L2, of the plunger shaft, where L2<L1,
wherein the total liquid volume additionally includes a priming liquid volume for priming an infusion tube when the infusion tube is connected to the syringe, and
wherein the value of L2 is selected to enable an additional relative movement between the plunger shaft and the barrel in order to prime the infusion tube with the priming liquid volume when the liquid volume in the barrel is reduced from the total liquid volume to the infusion liquid volume.

11. The device as in claim 10, wherein the first member and the second member are separate objects.

12. The device as in claim 10, wherein the first member and the second member are one object comprising the first and second elongated chambers.

13. The device as in claim 10, wherein the first member and the second chamber have, respectively, a length equal to L1 and L2.

14. The device as in claim 10, wherein the first member and the second member have a same length, L3, and the first elongated chamber and the second elongated chamber have, respectively, a length equal to L1 and L2.

15. The device as in claim 10, wherein each of the first member and the second member is a hollow cylinder including a base on one end and an opening opposite said base, the first member and the second member configured to receive, through the respective opening, the plunger shaft but not the barrel.

16. The device as in claim 10, wherein each of the first member and the second member has a transverse cross-sectional area selected from the group consisting of a ring-like cross-sectional area and a non-ring like shaped cross-sectional area.

17. (canceled)

18. The device as in claim 16, wherein the non-ring like cross-sectional area is generally “U”-shaped.

19. The device as in claim 10, further comprising a base member, wherein the first member and the second member outwardly protrude from the base member.

20. The device as in claim 19, wherein each of the first member and the second member comprises a base which is part of or attached to the base member, and an opening which is opposite the base and configured to receive the plunger but not the barrel.

21. The device as in claim 10, wherein the value of L1 is selected such that moving the syringe barrel relative to the plunger shaft when the plunger shaft resides in the first elongated chamber would result in the barrel containing the total liquid volume, and the value of L2 is selected such that further moving the barrel relative the plunger shaft when the plunger shaft resides in the second elongated chamber would result in the priming of the infusion tube with the priming liquid volume while leaving in the barrel only the infusion liquid volume intended for infusion.

22.-24. (canceled)

25. The device according to claim 2, wherein the values of L1 and L2 are calculated based on one or more parameters selected from the group of parameters consisting of:
(1) a maximum volume of liquid that the syringe barrel can contain,
(2) a volume of liquid required to fill the entire infusion tube;
(3) a volume of liquid to be delivered to the subject;
(4) a size of the syringe barrel;
(5) total length and inner diameter of the infusion tube;
(6) a cross-sectional area of the syringe barrel; and
(7) an internal diameter of the syringe barrel.

26. The device as in claim 25, wherein a length difference, Lp (where Lp=L1−L2) corresponds to the priming liquid volume that is required to fill up the entire infusion tube.

27. The device as in claim 26, wherein the value of Lp is selected from the range 0.36 mm-3.0 mm.

28.-30. (canceled)

31. The device as in claim 25, wherein L1 and L2 are adjustable to suit a variety of syringe barrels.

32. A method of operating a syringe liquid adjustment device to adjust a liquid volume in a barrel of a syringe, the syringe barrel initially containing an initial volume of liquid, the method comprising:
using a first elongated chamber of the syringe liquid adjustment device to reciprocally move the syringe barrel relative to the plunger shaft to reduce the amount of liquid in the syringe barrel from the initial liquid volume to a total liquid volume including an infusion liquid volume and a priming liquid volume; and
using a second elongated chamber of the syringe liquid adjustment device to reciprocally move the syringe barrel further relative to the plunger shaft to reduce the amount of liquid in the syringe barrel from the total liquid volume to the infusion liquid volume while priming an infusion tube with the priming liquid volume.

33. The method as in claim 32, wherein the step of using the second elongated chamber of the syringe liquid adjustment device to reciprocally move the syringe barrel further relative to the plunger shaft comprises removing the plunger shaft from the first elongated chamber and connecting the infusion tube to the syringe.
